(19) United States
(12) Patent Application Publication
(10) Pub. No.: US 2022/0380284 A1
(43) Pub. Date: Dec. 1, 2022

(54) SOLID FORMS OF DIMETHOXYPILLAR[5]ARENE (DMP5): HYDROCARBON FUEL UPGRADING AND GAS SORPTION

(71) Applicant: Georgetown University, Washington, DC (US)

(72) Inventors: K. Travis Holman, McLean, VA (US); Prashant K. Pandey, Washington, DC (US)

(73) Assignee: Georgetown University, Washington, DC (US)

(21) Appl. No.: 17/827,102

(22) Filed: May 27, 2022

Related U.S. Application Data
(60) Provisional application No. 63/194,637, filed on May 28, 2021.

(51) Int. Cl. C07C 43/205; B01J 20/22; B01J 20/30

(52) U.S. Cl. CPC C07C 43/205; B01J 20/22; B01J 20/3085; C07B 2200/13

(57) ABSTRACT

A composition comprising a compound of formula I:

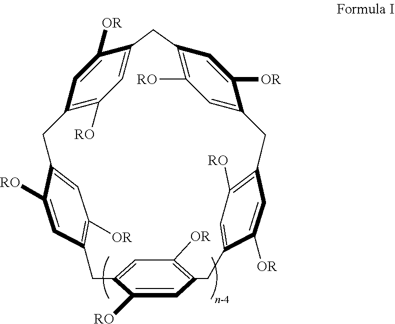

wherein n is 5, and R is methyl; and
the composition is in an essentially guest-free solid form.

### CROSS REFERENCE TO RELATED APPLICATION

This application claims the benefit of the earlier filing date of U.S. Provisional Application No. 63/194,637, filed on May 28, 2021, the entirety of which is incorporated herein by reference.

### ACKNOWLEDGMENT OF GOVERNMENT SUPPORT

This invention was made with government support under grant number DMR-1610882 awarded by the National Science Foundation. The government has certain rights in the invention.

### BACKGROUND

Porous molecular solids (PoMoS) are discrete-molecule materials whose microporous (or mesoporous) structures are not sustained by traditional chemical bonds, such as covalent or coordinate-covalent bonds. As such, they offer several advantages over porous network solids i.e., zeolites, porous carbons (and other inorganic porous materials), metal organic frameworks (MOFs), covalent organic frameworks (COFs), and porous polymers. For instance, PoMoSs derived from shape-persistent molecules that possess an innate cavity (or pore/window) can be among the more chemically and thermally stable porous materials. Pore stability arises from the fact that the most thermodynamically stable, “as-close-packed-as-possible” crystal structures of such compounds are essentially incollapsible and intrinsically porous. Chemical stability derives from the nature of the chemical bonds that sustain the molecular structure. Moreover, PoMoSs are typically soluble compounds and the “synthesis” of the porous structure can be as straightforward as dissolution and solvent evaporation. PoMoSs also offer the ability to mix and match components, allowing for easy pore functionalization or tuning. Thus, shape-persistent molecules that pack inefficiently, including porous organic or metal-organic cages, calixarenes and cavitands, cryptophanes, cucubiturils, and other organic and metal-organic macrocycles have recently received as much attention as PoMoSs.

### SUMMARY

Disclosed herein is a composition comprising a compound of formula I:

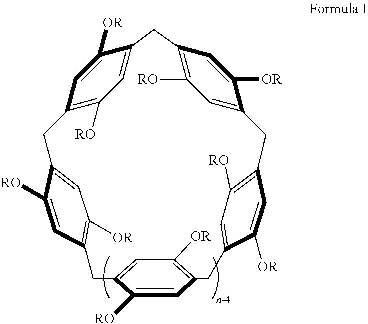

wherein n is 5, and R is methyl; and

the composition is in an essentially guest-free solid form.

Also disclosed herein is a composition comprising a host-guest complex, wherein the host is a compound of formula I:

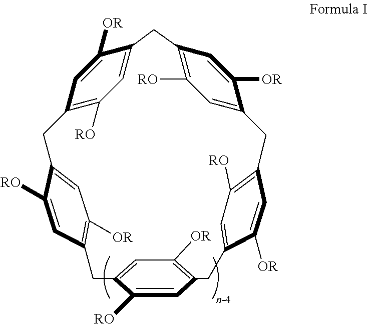

wherein n is 5, and R is methyl; and

the guest is molecules or atoms that exist as gasses at room temperature and atmospheric pressure.

Further disclosed herein is a method comprising:

exposing a sample comprising a chemical mixture to a composition; and

selectively forming a host-guest complex between the composition and one or more of the components from the sample, wherein

the composition comprises a compound of formula I:

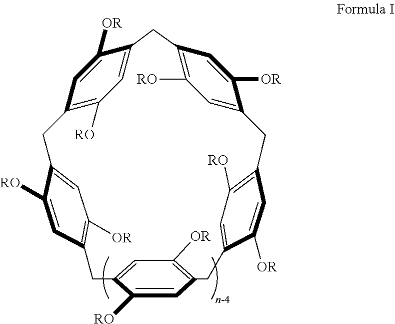

wherein n is 5, and R is methyl; and

the composition is in an essentially guest-free solid form.

Additionally disclosed herein is a method comprising:

exposing a liquid petroleum mixture to a composition; and

selectively forming a host-guest complex between the composition and one or more components of the liquid petroleum mixture, wherein

the composition comprises a compound of formula I:

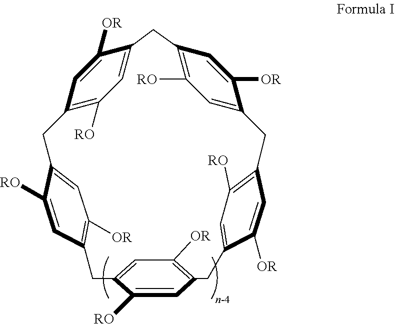

wherein n is 5, and R is methyl; and

the composition is in an essentially guest-free solid form.

The foregoing will become more apparent from the following detailed description, which proceeds with reference to the accompanying figures.

### BRIEF DESCRIPTION OF THE DRAWINGS

FIG. 1 is a scheme showing the structure of dimethoxypillar[5]arene (DMP5).

FIG. 2. A layer of the crystal packing of the known isostructural β-phase solvates of DMP5: MeCN@DMP5, EtOAc@DMP5, (CH₃)₂CO@DMP5 and CH₂Cl₂@DMP5. The solvent guests have been omitted and the occupied cavities are depicted in orange.

FIGS. 3A-3C. (FIG. 3A) TGA (1° C./min) of the known β-MeCN@DMP5 solvated form of DMP5. (FIG. 3B) DSC enthalpogram (endothermic, down) of β-MeCN@DMP5. (FIG. 3C) Hot stage optical microscopic analysis of crystals of β-MeCN@DMP5. The DSC enthalpogram and optical observation of bubbles evolving from the melt at 220° C. indicate that the compound can melt before all of the MeCN is lost.

FIGS. 4A-4C. (FIG. 4A) TGA of a-DMP5. (FIG. 4B) DSC enthalpogram of a-DMP5 with new crystalline guest-free DMP5 phases indicated. (FIG. 4C) PXRD pattern of a-DMP5 obtained from slow cooling of the melt.

FIG. 5A-5B. (FIG. 5A) ¹H NMR spectrum (400 MHz) of a-DMP5 in CD₂Cl₂ demonstrating the spectroscopic purity of the sample and absence of included solvents. (FIG. 5B) ¹³C NMR spectrum (100 MHz) of a-DMP5 in CD₂Cl₂ demonstrating the spectroscopic purity of the sample and absence of included solvents.

FIG. 6. Room temperature PXRD pattern of α-DMP5 (DUO).

FIG. 7. Room temperature synchrotron (λ=0.412764 Å) PXRD pattern of γ-DMP5 (APS).

FIG. 8. Thermal ellipsoid plot of the DMP5 molecule from the single crystal structure determination of α-DMP5 at 100 K illustrating the “collapsed” conformation of the DMP5 molecule in the guest-free α-DMP5 phase. Disorder of one of the dimethoxyphenyl groups is omitted for clarity.

FIGS. 9A-9B. A typical CO₂ absorption study for DMP5. (FIG. 9A) PXRD patterns of a) a-DMP5 starting material, b) a-DMP5 pressurized with 34 bar CO₂ in the environmental gas cell. The pattern was collected within minutes of initial exposure to CO₂ and shows complete conversion of a-DMP5 to β-xCO₂@DMP5. c) β-xCO₂@DMP5 after 60 days at ambient conditions (room temperature, ambient pressure, open air). The relative intensities of the two reflections near 9° 2θ serve as an indication of the relative CO₂ content (x). d) β-xCO₂@DMP5 after for 90 days at ambient conditions (x≈0.27). Tick marks represent the calculated (hkl) peak positions corresponding to a β-guest@DMP5 phase. (FIG. 9B) TGA analyses of the samples characterized by PXRD, illustrating the substoichiometric (x<1) CO₂ content of β-xCO₂@DMP5 (x<1) after storage under ambient conditions. Immediately after release of CO₂ pressure, the material contains about 0.82 CO₂ per DMP5. The loading decreases to 0.37 CO₂ and 0.27 CO₂ per DMP5 after 60 and 90 days, respectively.

FIG. 10. Room temperature CO₂ sorption and desorption isotherms of close-packed a-DMP5 and partially occupied, microporous β-0.16CO₂@DMP5.

FIGS. 11A-11C. Thermal ellipsoid plot of the β-1.0CO₂@DMP5 crystal structure at 100 K. The Fₒ-F_{c} map at the 0.45 e⁻/ Å³ surface is shown in green, illustrating the electron density associated with included CO₂. Crystal structure of β-Xeₙ@DMP5 (n≈1.4) (FIG. 11C) FIGS. 12A-12B. PXRD analysis of the DMP5 materials obtained after slurrying a-DMP5 in the various isomers of hexane. Reference patterns of a-DMP5, α-DMP5 and β-guest@DMP5 are shown for comparison.

FIGS. 13A-13B. (FIG. 13A) GC-MS analysis of 22DMB, 23DMB, 3 MP, 2 MP, and HEX, showing their characteristics retention times. The bottom chromatogram corresponds to the product isolated after slurrying a-DMP5 in an equal-volume mixture of the five isomers for 10 minutes. Only the low-value isomers HEX and a trace amount of 2 MP are absorbed by the DMP5. (FIG. 13B) GC-MS analysis of the DMP5 solid obtained after treating a sample of regular octane gasoline with a-DMP5. The characteristic peaks for PENT and HEX illustrate the ability of DMP5 to extract the low-octane paraffins directly from refined gasoline.

FIGS. 14A-14C. Crystal structures of β-HEX@DMP5, β-PENT@DMP5 and β-n-butane@DMP5.

FIG. 15. Room temperature PXRD pattern of γ-DMP5 (DUO).

FIG. 16. DSC enthalpogram of essentially pure, crystalline, guest-free α-DMP5.

FIG. 17. DSC enthalpogram of essentially pure, crystalline, guest-free γ-DMP5. Concomitant with melting of γ-DMP5 (onset around 155° C.), the sample crystallizes as α-DMP5, followed by eventual melting of the α-DMP5 form.

### DETAILED DESCRIPTION

### Overview

Disclosed herein are three solid forms of guest-free dimethoxypillar[5]arene (DMP5)—a cheap, shape-persistent macrocyclic compound available in a high-yielding single step from commodity chemicals—which are isolated for the first time, namely amorphous a-DMP5 and crystalline α-DMP5 and γ-DMP5. All previously reported structurally characterized solid forms of DMP5 are crystalline inclusion compounds wherein a solvent or other molecule or ion is included into the DMP5 cavity and/or solid-state structure (see CSD reference codes: AKEBEA, AKEBIE, AMOXUX, DACSOR, FIWMEF, FIWMIJ, FIWMOP, FOXFUW, JUGKUU, JUGLIJ, JUGLAB, JUGLEF, JUGLOP, JUGLUV, JUGMAC, LIKZIQ, MOCMIB, OQIKIK, OQISOY, OQISEU, PEHLOG, QIRVOF, QISPOA, QISPUG, QISQAN, QISQER, QISQIV, QISQOB, QISQUH, QISRAO, QISRES, SARXUG, VUFPEU). Amorphous DMP5 (a-DMP5) is novel in that it is a non-crystalline—as established by its X-ray diffraction pattern and differential scanning calorimetry trace-solid form of DMP5 that is essentially guest-free. Both α-DMP5 and γ-DMP5 are novel in that they are essentially pure crystalline forms of essentially guest-free DMP5, being characterized by their unique powder X-ray diffraction (PXRD) patterns, unit cells, and DSC enthalpograms.

As used herein, “crystal”, “crystalline” or “amorphous” refer to that characterized by a powder (PXRD) or single crystal X-ray diffraction (SCXRD) pattern. Those skilled in the art of X-ray diffraction are capable of understanding that the experimental error depends on instrumental conditions, sample preparation and sample purity. In particular, it is well known to those skilled in the art that X-ray diffraction patterns may change with the changes of instrumental and sample conditions. It needs to be particularly pointed out that the relative intensities of PXRD peaks may also change with the changes of experimental conditions, so the relative peak intensities should not be considered as the only or conclusive factor. Additionally, experimental errors in the angles of PXRD peaks observed can vary and these errors should also be considered, and usually differences within +0.2 two-theta are allowed. Additionally, experimental factors such as sample position may lead to overall PXRD peak shifts, and usually a certain shift is allowed. Therefore, those skilled in the art are capable of understanding that any crystalline forms having the same or similar characteristic PXRD peaks as those disclosed herein are within the scope of the presently disclosed subject matter. Additionally, those skilled in the art will understand that unit cell determinations by SCXRD and PXRD are susceptible to experimental error and temperature effects (thermal expansion). Additionally, those skilled in the art of X-ray diffraction are capable of using the unit cell information disclosed herein to calculate a PXRD pattern for comparison with an experimental PXRD pattern to determine, after considering additional factors (e.g., composition) and allowing for a reasonable degree of instrumental error, whether a sample contains one or more of the forms of DMP5 within the scope of the presently disclosed subject matter.

Crystalline forms as disclosed herein are pure and essentially free of any other crystalline forms. For example, when “essentially free of” is used for describing a novel crystalline form, it means that the content of other crystalline forms in the novel crystalline form is less than 10% (mol/mol), more specifically less than 5% (mol/mol), and furthermore specifically less than 1% (mol/mol).

Guest-free DMP5 is a highly selective sorbent for capturing gases (e.g., CO₂, xenon and n-butane) or extracting low value linear paraffins (e.g., n-hexane, n-pentane, monomethyl paraffins) directly from relevant liquid petroleum mixtures such as gasoline and isomeric hexane mixtures. The crystal structures of α-DMP5, β-xCO₂@DMP5, β-n-hexane@DMP5, β-n-pentane@DMP5, and β-n-butane@DMP5 are described.

### Compounds, Compositions and Methods of Use

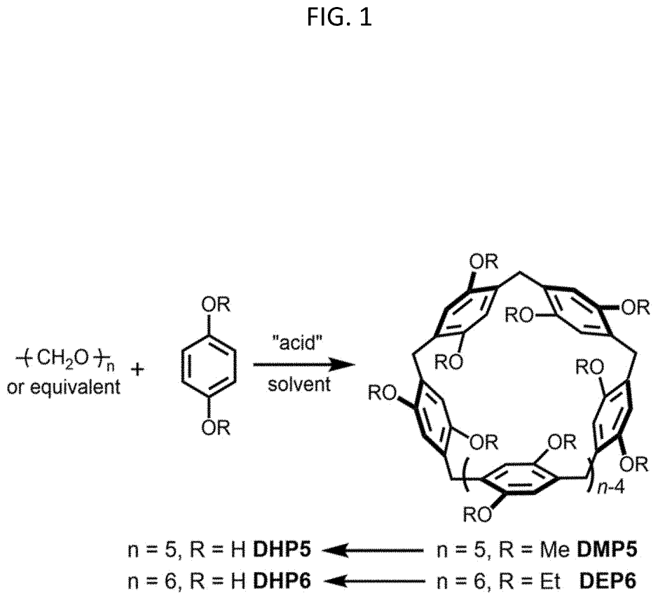

A promising addition to the family of intrinsically PoMoSs are the shape-persistent, tubular pillar[n]arene (Scheme 1, n=5-15) macrocyles, first introduced with the discovery of dimethoxypillar[5]arene (DMP5) by Ogoshi et al, J. Am. Chem. Soc. 2008, 130, 5022. A plethora of functionalized pillar[n]arenes have since been synthesized for various applications. For potential applications as commodity-scale sorbents, however, the smaller, early stage (R=alkyl/hydroxy) pillar[n]arenes (n=5, 6) offer the greatest promise due to their ease of synthesis and relatively low cost. For instance, DEP6, DHP6, DHP5, (FIG. 1) have all been explored as porous materials for chemical separations.

It is surprising that DMP5 has so far been the least studied in the context of porosity and its solid-state chemistry, considering that it is available in the highest yield (up to 81% reported), in a single step, from the commodity chemicals p-anisole and formaldehyde. Indeed, though a variety of DMP5 solvates have been reported, there are no reports on the crystal/solid forms of pure, guest-free DMP5. Tan et al., Adv. Mater. 2014, 26, 7027 showed that dihydroxypillar[5]arene (DHP5)—a derivative of DMP5—functions as a PoMoS for selective CO₂ capture at room temperature, but purported that “DMP5” takes up almost no CO₂ at 298 K. Yet their data suggest that the DMP5 material studied still contained included solvents/guests.

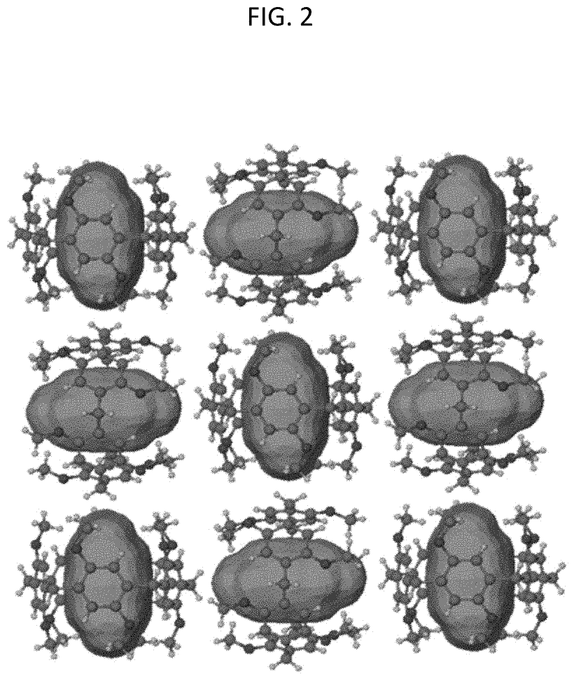

Several small molecule solvates of DMP5 (MeCN, EtOAc, (CH₃)₂CO, and CH₂Cl₂; (CSD reference codes: FIWMEF, FIWMIJ, FIWMOP, MOCMIB)) crystallize in an isostructural fashion, as a tetragonal (I4₁/a) “β”-phase, exhibiting a herringbone type packing of the host. A notable feature of these β-DMP5 clathrates is the presence of discrete (non-interconnected) cavities—or zero-dimensional (0D) pores—offered by the tubular host, and the simple occupation of these cavities by the encapsulated solvents (FIG. 2). DMP5 was therefore explored in the context of the possibility of forming a guest-free, 0D porous “beta-not” β0-DMP5) phase. The results are described herein, including the first preparation and characterization of the solid forms of guest-free DMP5, including an amorphous form (a-DMP5) and two crystalline forms (α-DMP5 and γ-DMP5). Upon application of moderate pressures of CO₂, all three guest-free forms of DMP5 transform to crystalline β-xCO₂@DMP5 (x≈0.16-1.3), the first reported gas-occupied form of DMP5. Thus, contrary to published literature, DMP5, in guest-free form, does function as effective sorbent for the uptake/storage of gases. The crystal structure of xCO₂@DMP5 has been determined and is shown to isostructural to the R-form solvates. “Partially emptied” forms of xCO₂@DMP5 (x<1) were also discovered to maintain a formally 0D microporous β-phase structure that can absorb CO₂ at room temperature in amounts comparable to that observed by dihydroxypillar[5]arene (DHP5). Guest-free DMP5 acts as a highly selective sorbent for normal linear and methyl-branched hexane isomers. It transforms to the corresponding β-form solvates upon exposure to these compounds and selectively extracts n-hexane and methyl-pentane isomers from a mixture of isomeric hexanes. Direct treatment of commercial gasoline with guest-free DMP5 results in the extraction of certain low-value, low octane number linear and methyl-branched paraffins, thus illustrating the potential of guest-free DMP5 for gasoline upgrading.

Also disclosed herein are methods of using DMP5 and DMP5 compositions for certain industrial applications. Crystalline DMP5s may have characteristics, such as crystalline packing, that have not been observed in other DMPs. Additionally, the crystalline DMP5s may comprise void spaces within the crystalline structure.

A void space maybe a pore or cavity within the crystalline DMP5 and DMP5 compositions. The void spaces within the crystalline structure may be empty, i.e., free of any molecules or atoms. In certain embodiments, the voids that are free of solvent molecules. The void spaces within the crystalline composition may also comprise gas molecules or atoms, where a gas is defined as an atom or molecule that is normally in its gas phase at standard temperature and pressure conditions.

Also disclosed herein are compositions comprising solid forms of DMP5 that further comprise guest molecules that may be complexed in the cavities of the DMP5 to form host-guest complexes.

In certain embodiments, the guest molecule may be a gas molecule. Illustrative gas molecules include acetylene, argon, krypton, xenon, radon, carbon dioxide, methane, ethylene, ethane, propyne, propene, propane, butanes, butenes, butadienes, fluoromethane, chloromethane, chloroethane, dimethylether, freons, gaseous fluorocarbons, methanethiol, oxygen, nitrogen, and bromomethane.

In certain embodiments, the guest gas is selected from one or more C₁ hydrocarbon gasses, C₂ hydrocarbon gasses, C₃ hydrocarbon gasses, and C₄ hydrocarbon gasses.

In certain embodiments, the guest gas is a noble gas. In certain embodiments, the guest gas is argon, krypton, xenon, or radon.

The compounds and compositions may be characterized by their ability to selectively complex gas molecules. This property is useful for gas separations, wherein two or more gasses may need to be separated in, for example, an industrial process.

Certain compositions disclosed herein are capable of forming a host-guest complex with one or more guest gas molecules within its cavities; wherein the guest gas is selected from one or more C₁ hydrocarbon gasses, C₂ hydrocarbon gasses, C₃ hydrocarbon gasses, and C₄ hydrocarbon gasses.

In an embodiment, the composition is characterized by forming a host-guest complex with one or more C₁ hydrocarbon gases selectively over one or more C₂ hydrocarbon gasses, C₃ hydrocarbon gasses, and/or C₄ hydrocarbon gases. In another embodiment, the composition is characterized by forming a host-guest complex with one or more C₂ hydrocarbon gases selectively over one or more C₁ hydrocarbon gasses C₃ hydrocarbon gasses, and/or C₄ hydrocarbon gases. In another embodiment, the composition is characterized by forming a host-guest complex with one or more C₃ hydrocarbon gases selectively over one or more C₁ hydrocarbon gasses, C₂ hydrocarbon gasses, and/or C₄ hydrocarbon gases. In a further embodiment, the composition is characterized by forming a host-guest complex with one or more C₄ hydrocarbon gases selectively over one or more C₁ hydrocarbon gasses, C₂ hydrocarbon gasses, and/or C₃ hydrocarbon gases.

In certain embodiments, the C₁ hydrocarbon gas is methane, the C₂ hydrocarbon gas is selected from one or more of ethane, ethylene, and acetylene, the C₃ hydrocarbon gas is selected from one or more of propane, propene, and propyne, and the C₄ hydrocarbon gas is selected from one or more of butane, butene and butyne.

In an embodiment, the composition is capable of forming a host-guest complex with one or more guest gas molecules within its cavities, wherein the one or more guest gas molecule(s) is/are selected from CH₃C₁ and CH₃CH₂C₁, and wherein the composition is characterized by selectively forming a host-guest complex with CH₃C₁ over CH₃CH₂C₁ or CH₃CH₂C₁ over CH₃C₁.

In another embodiment, the composition is capable for forming a host-guest complex with guest gas molecules within its cavities, wherein the one or more guest gas molecule(s) is/are selected from CH₃C₁ and CH₃OCH₃, and wherein the composition is characterized by selectively forming a host-guest complex with CH₃C₁ over CH₃OCH₃ or CH₃OCH₃ over CH₃C₁.

In another embodiment, the composition is capable for forming a host-guest complex with guest gas molecules within its cavities, wherein the one or more guest gas molecule(s) is/are selected from the Ar, Kr, Xe, or Rn, and wherein the composition is characterized by selectively forming a host-guest complex with one of Ar, Kr, Xe, or Rn over the others.

In certain embodiments, the DMP5-gas, host-guest complex compositions may also be used for the confinement of gases at ambient temperatures that are at least the boiling points of the gases. In an embodiment, the ambient temperature is at least 10° C. greater than the boiling point of the gas. In another embodiment, the ambient temperature is room temperature or about ° C. The property of gas confinement has particular utility for the separation of gases as well as the confinement and storage of gasses. In a particular embodiment, the confined and or separated gasses may be radioactive gasses. In an embodiment, the radioactive gas may be a radioactive isotope of xenon (Xe) and/or krypton (Kr).

Also disclosed herein are methods of using DMP5s and DMP5 compositions for industrial purposes, including, but not limited separating gas mixtures, separating liquid petroleum mixtures such as, for example, gasoline or separating isomeric hexane mixtures. In an embodiment, compositions comprising DMP5s may be used to separate one or more gasses from other gasses. In another embodiment, compositions comprising the DMP5s may be used to separate one or more linear paraffins from liquid petroleum mixtures. Illustrative linear paraffins include n-hexane, n-pentane, n-heptane, and monomethyl paraffins (e.g., monomethyl substituted 2-methylpentane, 3-methylpentane, and 2-methylhexane).

The methods are generally useful for the separation and confinement of gasses. In an embodiment, the method is for gas separation comprising:
(i) exposing a sample comprising two or more gasses to a DMP5 solid form as disclosed herein; and
(ii) selectively forming a host-guest complex between the DMP5 solid form and one or more of the gasses from the sample.

Also disclosed herein are methods for the separating hydrocarbon gasses. As a non-limiting illustration, hydrocarbon gasses may be separated on the basis of length, i.e., number of carbon atoms, structure, i.e., straight chained versus branched, or saturation, i.e., the separation of alkanes, alkenes, and/or alkynes. In an embodiment, compositions of the invention are capable of doing separations of hydrocarbons including, but not limited to, C₁ hydrocarbons, C₂ hydrocarbons, and C₃ hydrocarbons.

In an embodiment, the method comprises separating propane and propene. In another embodiment, the method comprises separating ethylene and ethane. In another embodiment, the method comprises separating hexane (particularly n-hexane) and hexene.

Also disclosed herein are methods for separating gases containing functional groups. For example, the compositions may be used to separate haloalkanes or ethers that are normally in their gas phase at standard temperature and pressure. In an embodiment, the compositions are capable of separating dimethyl ether from chloromethane and/or chloroethane. In another embodiment, the compositions are capable of separating chloromethane and chloroethane.

Further disclosed herein is a method of gas storage comprising:
(i) exposing a sample comprising one or more gasses to an empty crystalline DMP5 composition, and
(ii) forming a host-guest complex between the composition and one or more gasses from (i) the gas;

wherein the complex is capable of retaining at least 95% of the gas at an ambient temperature that is at least 10° C. greater than the boiling point of the gas.

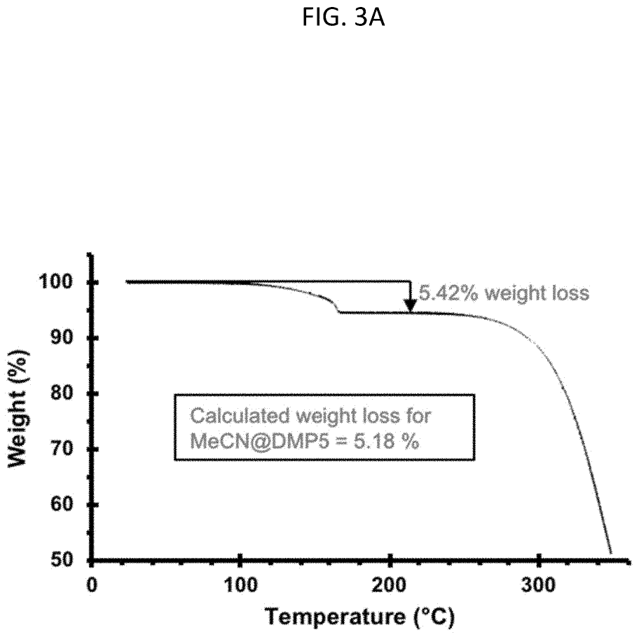

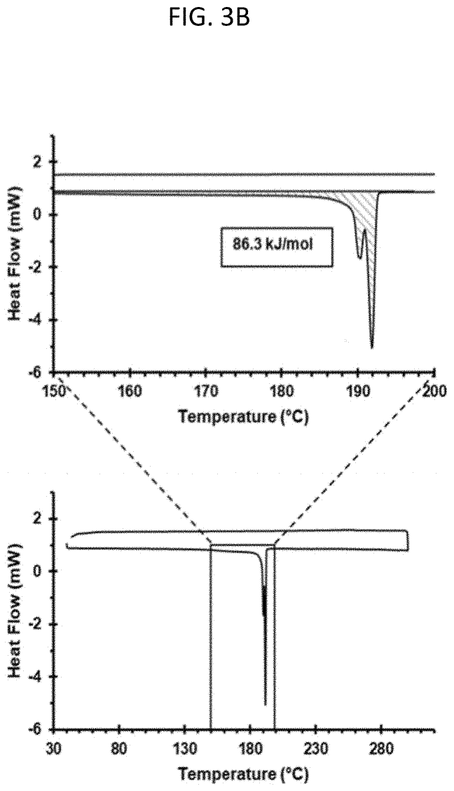

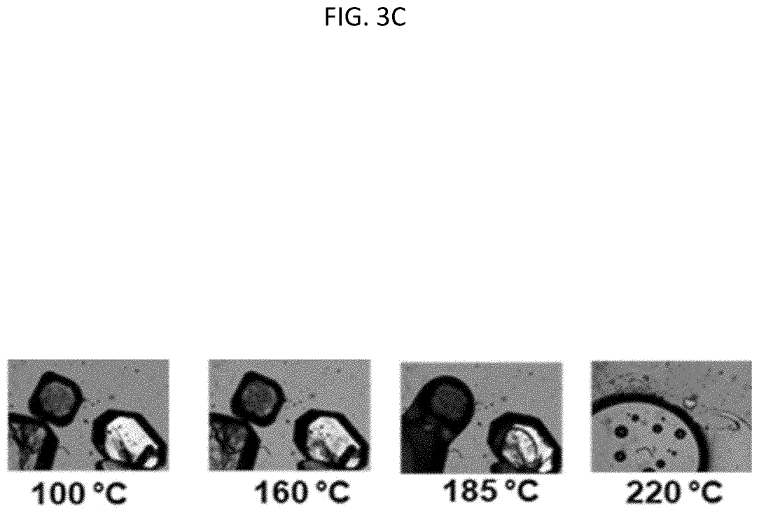

The reported syntheses of DMP5 yield, depending upon the workup, solvates or ill-defined mixed solvates in accord with the small molecule scavenging properties of the macrocyclic host. (Schonbeck et al., J. Phys. Chem. B 2015, 119, 6711) Repeated recrystallization of the product from hot MeCN gave us the 1:1 known solvate (hereafter β-MeCN@DMP5) in phase-pure form according to PXRD, TGA, and ¹H NMR spectroscopy. The high affinity of DMP5 for small solvent molecules makes preparation of activated/guest-free DMP5 rather challenging. Several others have purportedly used DMP5 in various experiments (e.g., for solution binding studies) but examination of the experimental data and procedures suggest that the activation procedures employed are either ill-defined or were unsuccessful at emptying the host. Thermal gravimetric analysis (TGA), differential scanning calorimetry (DSC), and hot stage microscopic analysis of β-MeCN@DMP5 (FIGS. 3A-3C) reveal that loss of MeCN begins just below 100° C., but can continue beyond the melting point of the material.

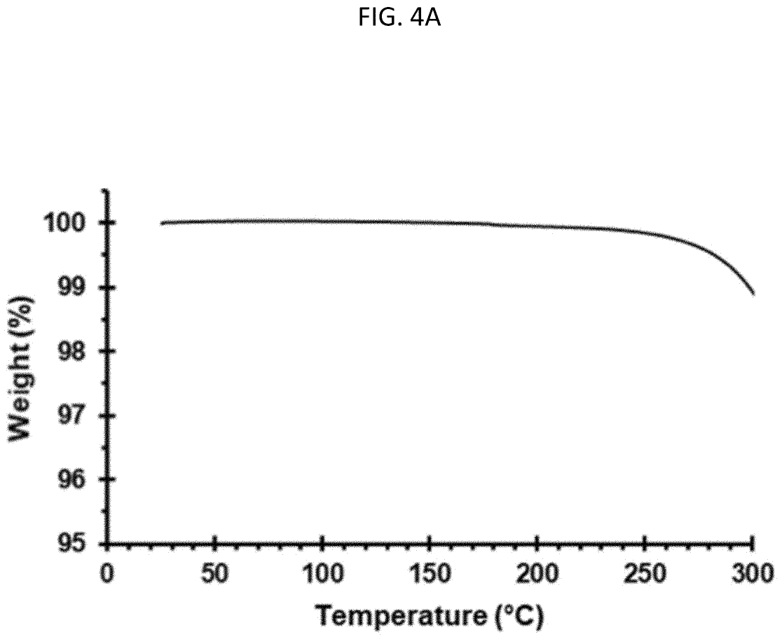

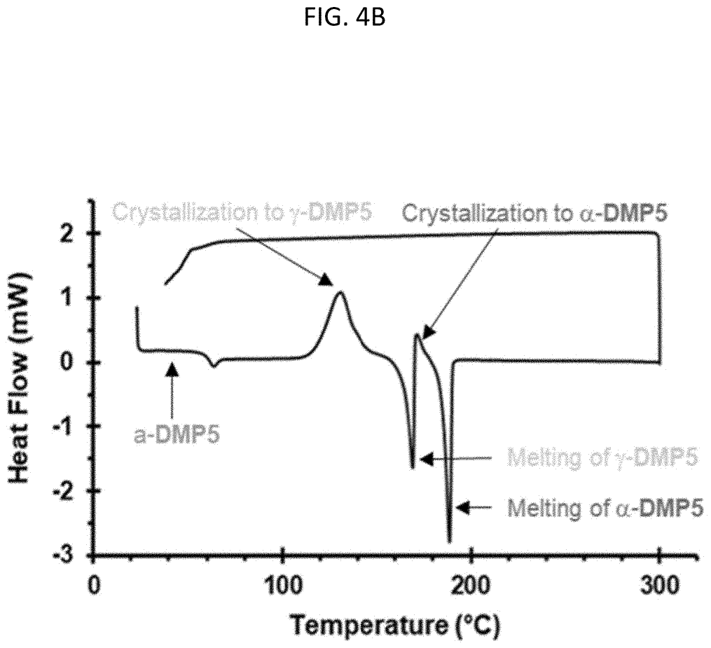

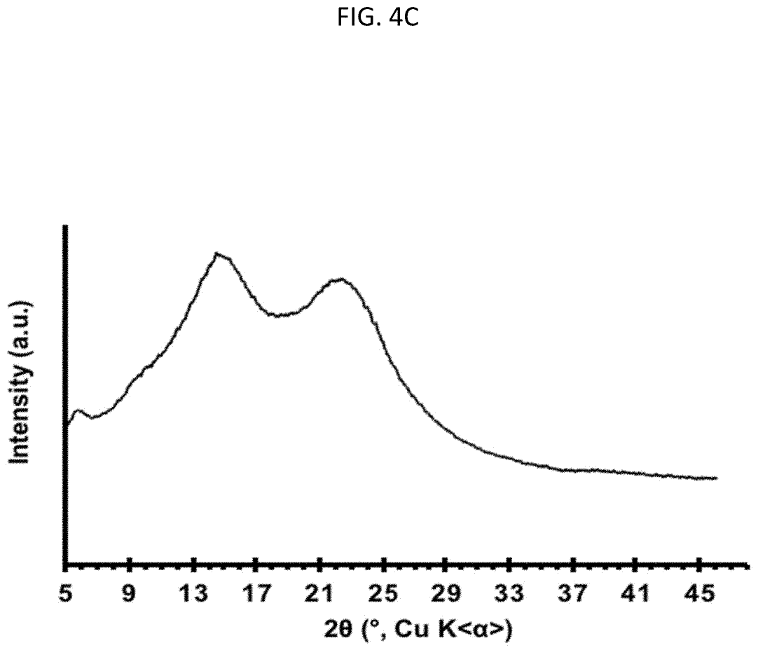

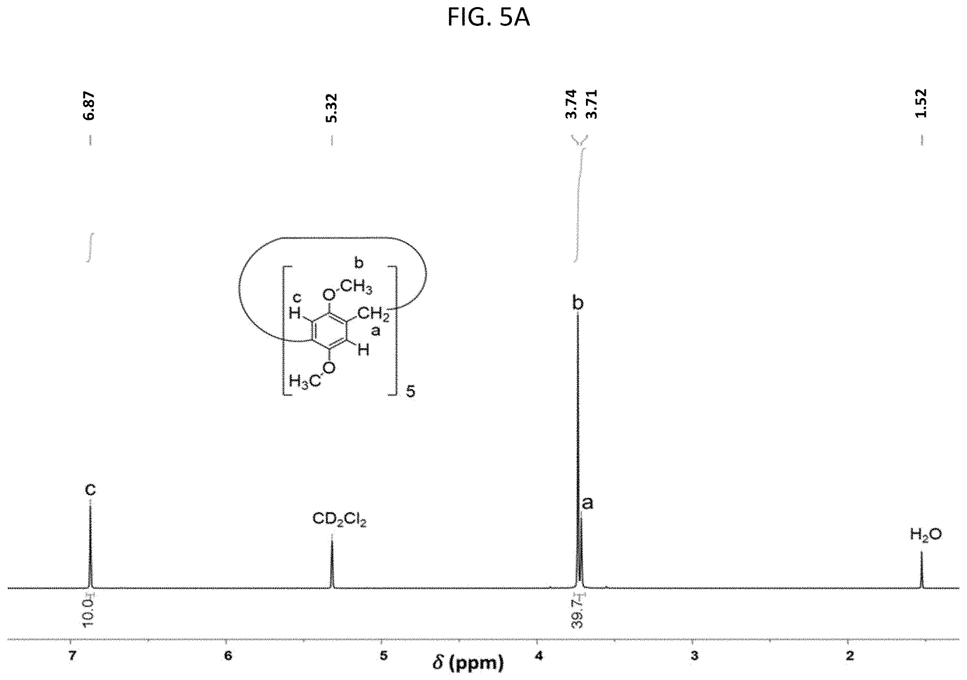

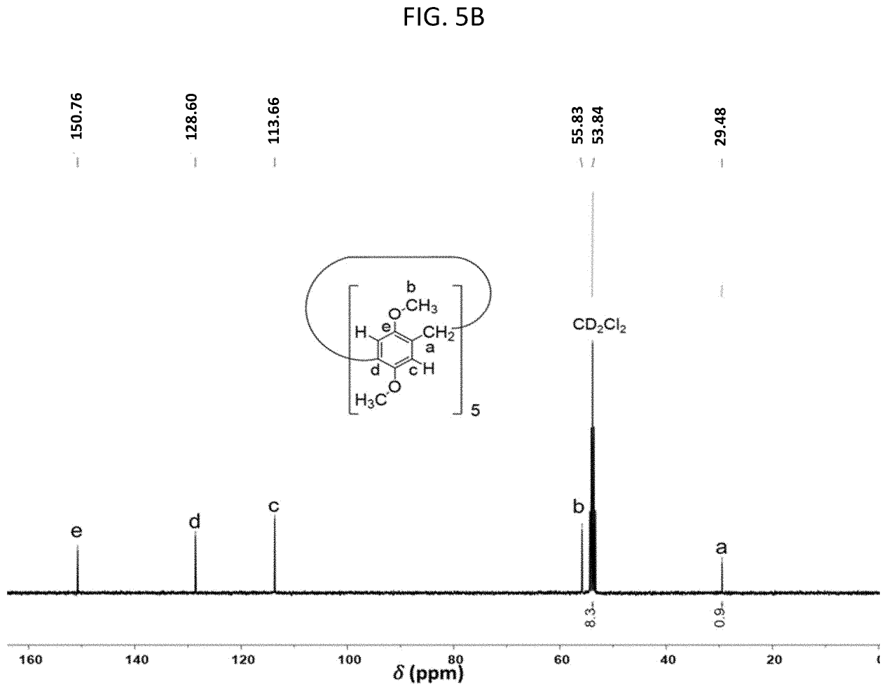

Slow cooling (2° C./min) of the pure (i.e., guest-free) DMP5 melt in the DSC enthalpogram from 300° C. to room temperature leads to an amorphous solid, hereafter a-DMP5. Similarly, a-DMP5 can be obtained by quench cooling of the pure molten form. FIGS. 4A-4C illustrate the TGA, DSC enthalogram, and powder X-ray diffraction (PXRD) pattern of a-DMP5. FIGS. 5A-5B provide the ¹H NMR and ¹³C NMR spectra of a-DMP5 taken in CD₂Cl₂, demonstrating the absence of MeCN.

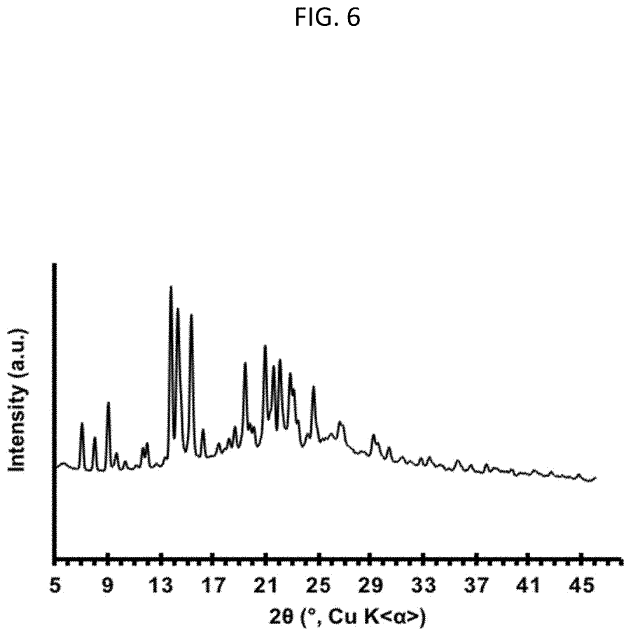

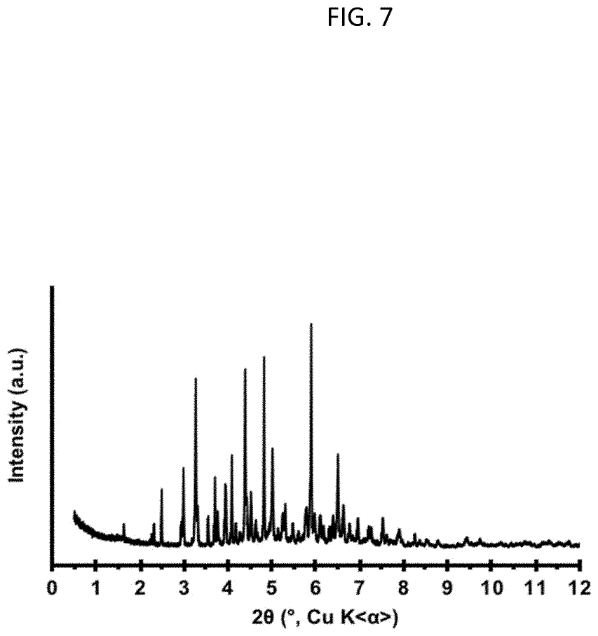

Slow cooling of molten DMP5 under vacuum (vacuum oven) yields either one of two crystalline guest-free DMP5 polymorphs, hereafter α-DMP5 or γ-DMP5. It was found that 7-DMP5 phase is susceptible to eventual solid-to-solid conversion to α-DMP5, demonstrating that α-DMP5 is the more thermodynamically stable form at room temperature. PXRD patterns of these phases are illustrated in FIGS. 6 and 7, respectively. The TGA, ¹H NMR, and ¹³C NMR characterization data corresponding to α-DMP5 or γ-DMP5 are essentially identical to that of a-DMP5. Slow heating of a-DMP5 will also yield crystalline α-DMP5 or γ-DMP5, depending upon conditions. DSC analysis of a-DMP5 (FIG. 3A) reveals what appears to be a glass transition (˜60° C.), followed by crystallization of γ-DMP5 (confirmed by PXRD analysis of a sample where the experiment was stopped after crystallization of γ-DMP5) that, upon further heating, melts (˜165° C.) and then immediately recrystallizes to the α-DMP5 polymorph. It is noteworthy that the melting point of pure α-DMP5 (191-192° C.) is much higher than that of γ-DMP5 (˜165° C.), suggesting a large difference in their lattice energies.

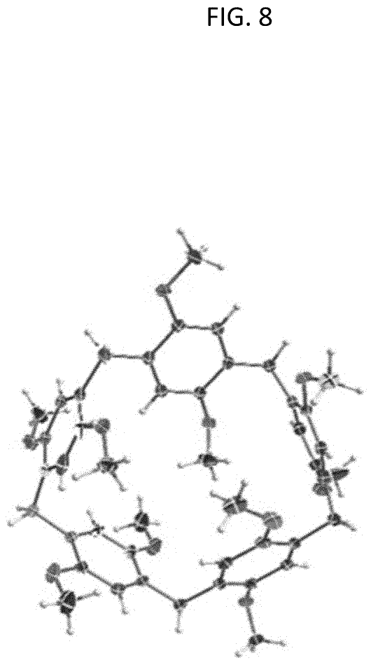

Given its nature as a concave, macrocyclic host and its shape-persistent structure, it was of interest to establish whether the previously known β-guest@DMP5 phases could be emptied to give a porous “βₒ” phase, or whether any of the three new guest-free forms (a-DMP5, α-DMP5, or γ-DMP5) were porous. A foreseeable challenge in studying the βₒ-DMP5 phase, however, is the removal of guest molecules without collapsing the host structure to the α-DMP5, or γ-DMP5 phases. Indeed, according to PXRD experiments, thermal removal of guest solvents from β-solvent@DMP5 under vacuum generally leads to conversion to α-DMP5. Single crystals of α-DMP5 were successfully obtained by vacuum sublimation of the desolvated material. The crystal structure of α-DMP5 was determined at 100 K by single crystal X-ray diffraction and a unit cell was determined at room temperature. α-DMP5 crystallizes in the triclinic P−1 space group with unit cell dimensions of approximately a=8.79 Å, b=12.82 Å, c=18.24 Å, α=96.1°, β=90.9°, γ=105.7° at 100 K At room temperature, the unit cell measures approximately a=8.86 Å, b=12.91 Å, c=18.03 Å, α=96.62°, β=90.86°, γ=105.29°. FIG. 8 shows the structure of the DMP5 molecule derived from the crystal structure at 100 K. A portion of the DMP5 molecule is found to be disordered such that one of the aryl groups resides in one of two orientations, related by an approximate 1800 rotation about the axis defined by the two adjacent methylne carbons. Perhaps surprisingly, the crystal structure of α-DMP5 was found to be essentially close-packed and non-porous. Unlike the familiar D₅-symmetric tubular conformation of the DMP5 molecule in the β-guest@DMP5 clathrates—wherein the methylene bridges of the macrocycle are coplanar and the dihedral angles between the plane of methylene bridges and the arene rings are all approximately 90°—the DMP5 molecule in the α-DMP5 phase exists in an essentially collapsed, low symmetry conformation, wherein each of the five methylene bridges of the macrocycle adopt an envelope-type arrangement reminiscent of cyclopentane and the cavity is essentially filled by the methoxy groups of arenes that have turned about their CH₂—Ar—CH₂ axes. Thus, it seems that the high temperature treatment necessary for desolvation of the β-guest@DMP5 phases leads to collapse of the β-DMP5 structure.

### Preparation and Characterization of β-xCO₂@DMP5 Gas Clathrates and Microporous β-xCO₂@DMP5 (x<1)

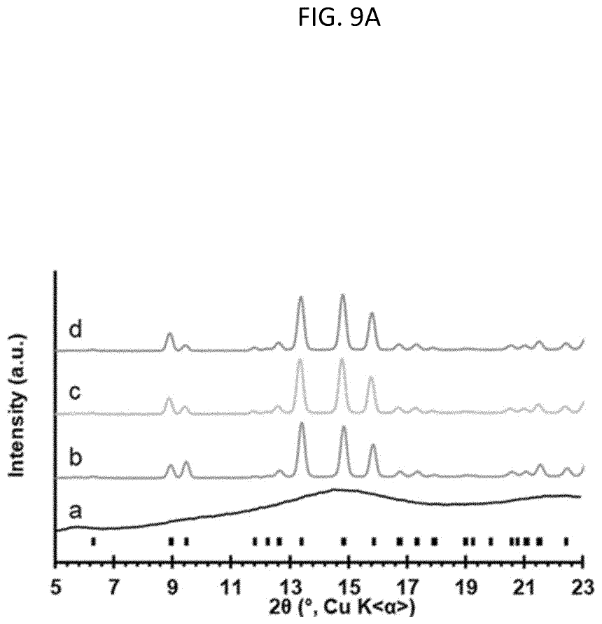

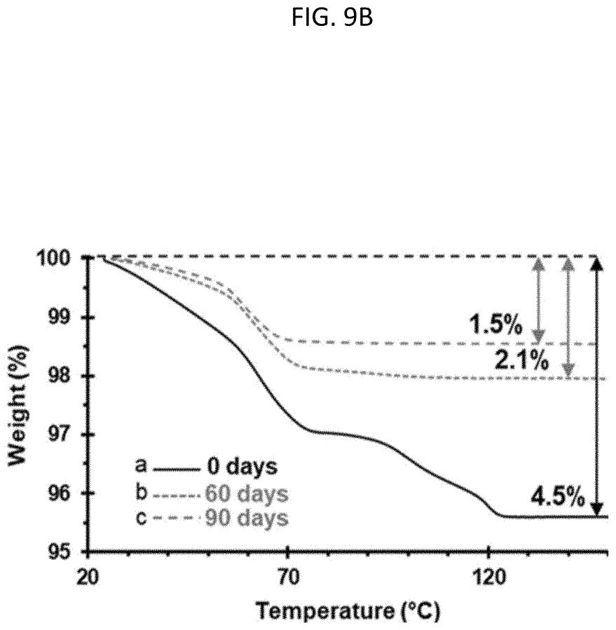

Seeking alternative means to generate a putative, 0D porous β₀-DMP5 phase by activation of a β-guest@DMP5 inclusion compound, we turned to the enclathration of a guest with a small kinetic diameter, presuming that the smaller molecule may be released at lower temperatures. Thus, CO₂ was explored as possible guest for DMP5. It was found that a-DMP5, α-DMP5, and γ-DMP5 could each be successfully converted to β-xCO₂@DMP5 by application of high pressures of CO₂ (34 bar). The PXRD patterns of the resulting, phase-pure β-xCO₂@DMP5 clathrates confirmed their β-phase structure (FIG. 9). Though a-DMP5 was almost instantly converted to β-xCO₂@DMP5 (minutes) upon pressurization, the conversion of α-DMP5 and γ-DMP5 to β-xCO₂@DMP5 was slower at room temperature. Clearly, though, under CO₂ pressure, the β-xCO₂@DMP5 phase is more stable than a-DMP5 or the collapsed α-DMP5 or γ-DMP5 phases. The initial total CO₂ content of the resulting β-xCO₂@DMP5 phases prepared under pressure was difficult to quantify. Upon release of the CO₂ pressure, the material spontaneously and rapidly undergoes CO₂ loss at room temperature. Immediate analysis of the samples by tandem TGA-MS and IR spectroscopy (2332 cm⁻¹) suggests at least 0.84 CO₂ molecules per DMP5. As CO₂ loss follows a deceleratory rate law, however, the samples were found to maintain a fractional amount of CO₂—that is, clearly substoichiometric occupancy of CO₂—and a phase-pure β-phase structure after several weeks of storage under ambient conditions (room temperature, atmospheric pressure, open container). For example, a sample of β-xCO₂@DMP5 left under ambient conditions for 90 days was found to maintain a phase-pure β-form structure with at most x=0.27 equivalents of CO₂ according to TGA analysis. Similarly, mild heating (30° C.) of freshly prepared β-xCO₂@DMP5 for one week yielded phase-pure β-0.23CO₂@DMP5, which, after further room temperature treatment under dynamic vacuum for 30 hours gave phase-pure β-0.16CO₂@DMP5. Eventually, however, once the CO₂ content fell below x≈0.15 or less, it was found that the material began to undergo conversion to the collapsed α-DMP5 form. That the β-xCO₂@DMP5 phase remains phase-pure, even with a clearly substoichiometric CO₂ content (x<<1 per host cavity), is particularly noteworthy and suggests that up to ˜84% of the DMP5 cavities in the β-0.16CO₂@DMP5 phase are empty. The partially occupied β-0.16CO₂@DMP5 material ought thereby to be functionally microporous.

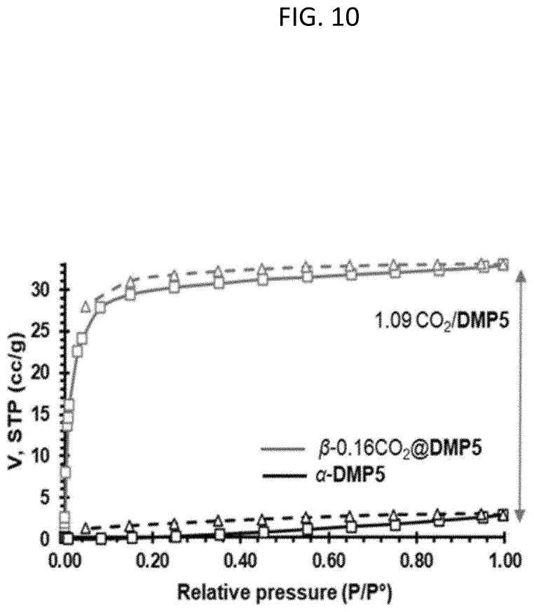

Remarkably, as shown in FIG. 10, the β-0.16CO₂@DMP5 material indeed proved to be porous, exhibiting a Type I low pressure sorption isotherm for uptake of CO₂ at room temperature. The material takes up about 31 cm³ CO₂(STP)/g at 1 bar, corresponding to an additional 1.09 equivalents of CO₂ or 58 (mgCO₂)/g. The total CO₂ capacity of DMP5 at 1 bar is therefore 1.25 equivalents or 67 (mgCO₂)/g. The behavior is in stark contrast to that of the three empty forms of DMP5 (a-DMP5, α-DMP5, or γ-DMP5), which show no evidence of porosity and exhibit essentially no CO₂ uptake under the same conditions. Similarly, the behavior of β-0.16CO₂@DMP5 is in contrast with that of the fully occupied, β-phase clathrate, β-guest@DMP5, which is also non-porous. In all, the data compare very favorably to the room temperature CO₂ capacity of dihydroxypillar[5]arene (DHP5) reported by Tan et al. (1.3 equivalents 88 (mgCO₂)/g, 1.34 equivalents).

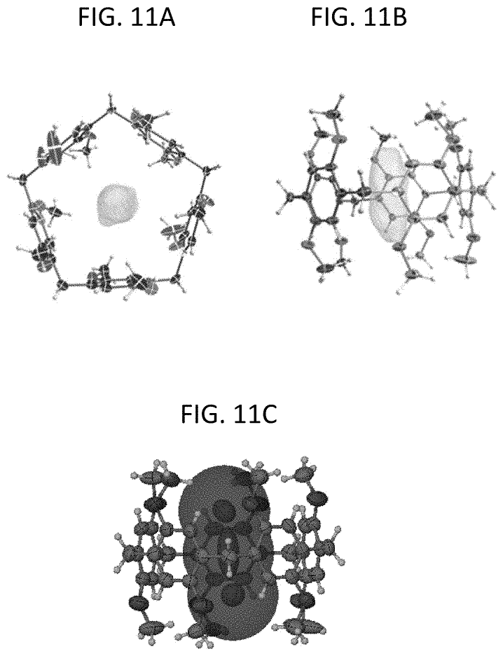

Single crystals of the fully occupied β-1.0CO₂@DMP5 clathrate were obtained by pressurizing a nearly saturated, warm (50° C.) solution of a-DMP5 in 2,6-dichlorotoluene with 34 bars of CO₂ for 7 days (while cooling to room temperature) using a custom-built, stainless steel pressure vessel. After removing the gas overpressure, a single crystal was rapidly mounted, straight from the mother liquor, onto the tip of a glass fiber and quickly loaded into the cold stream of the single crystal diffractometer for X-ray structure determination. Exposure of the crystals to atmospheric conditions for more than a few seconds resulted in their rapid deterioration into powder form due to rapid loss of the enclathrated CO₂. The crystal structure was determined by X-ray diffraction at 100 K. The β-1.0CO₂@DMP5 clathrate was found to be essentially isostructural to the aforementioned β-solvent@DMP5 solvates, crystallizing in the tetragonal I4₁/a space group with unit cell parameters (100 K) of about a=14.86 Å and c=38.95 Å. The enclathrated CO₂ molecule was found to be disordered, even at 100 K, but SQUEEZE analysis of the data revealed the total electron count within the DMP5 cavity to be 22 e⁻, corresponding to exactly one molecule of CO₂ per cavity. Thermal ellipsoid plots of the DMP5 host and the Fₒ-F_{c} difference electron density map are depicted in FIGS. 11A-11B.

### Selective Sorption of Normal and Monomethylated Paraffins.

Efficient separation of hexane, pentane, and heptane isomers remains a great challenge to the petroleum industry. In current petroleum refining technology, straight run gasoline contains appreciable quantities of low-value (low octane number) isomers of hexane (as well as heptane and pentane) that are mixed with the high-quality isomers (high octane number). Particularly egregious are the normal linear n-hexane (25)(HEX), n-pentane (62)(PENT) and n-heptane (0)(HEPT) and monomethyl substituted 2-methylpentane (75)(2 MP) and 3-methylpentane (75)(3 MP) and 2-methylhexane (52)(2MH) because of their relatively low octane numbers (shown in parentheses) and their volatility, affecting the Reid vapor pressure of the gasoline product. In current refining technology, achieving the desired octane number requires reforming of the linear and monomethyl paraffin isomers via isomerization catalysts, which, somewhat inefficiently, convert a portion of the linear paraffins into their more valuable (higher octane number), more branched isomers. For example, the 2,2-dimethyl butane (22DMB) and 2,3-dimethylbutane (23DMB) isomers of hexane have octane numbers of 92 and 100, respectively. Because of the inefficiency of the isomerization process, current technologies require energy intensive removal of the remaining linear and monomethyl substituted paraffins isomer from the product isomeric mixture so that they be recycled back into the isomerization reactor feed. Improvements in this process could have a significant positive impact on the associated monetary and environmental (energy consumption) costs.

Compared to existing porous materials such as molecular sieve zeolites, PoMoSs have the potential to more effectively separate molecules by shape. Moreover, they may be able to do this by acting directly on the liquid form of the sorbate as opposed to vapor phase. That the cavity of the β-DMP5 phase is highly complementary to short chain linear hydrocarbons is relatively underappreciated. Indeed, the measured binding constant of DMP5 for n-hexane (Kₐ=4 M⁻¹ in CDCl₃) initially suggests that DMP5 exhibits only low affinity for this compound. On the contrary, as described below, we have found that DMP5 exhibits a very high affinity for n-hexane and 2-methylhexane, and can extract these compounds and n-pentane directly from commercial regular octane gasoline.

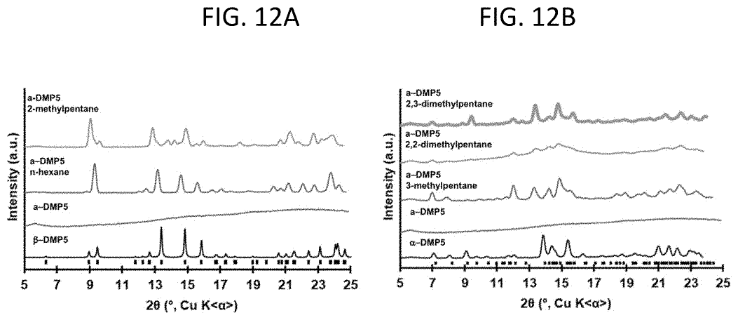

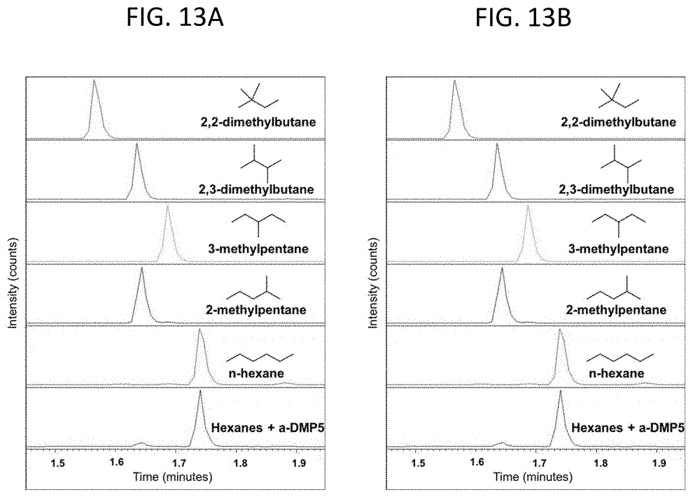

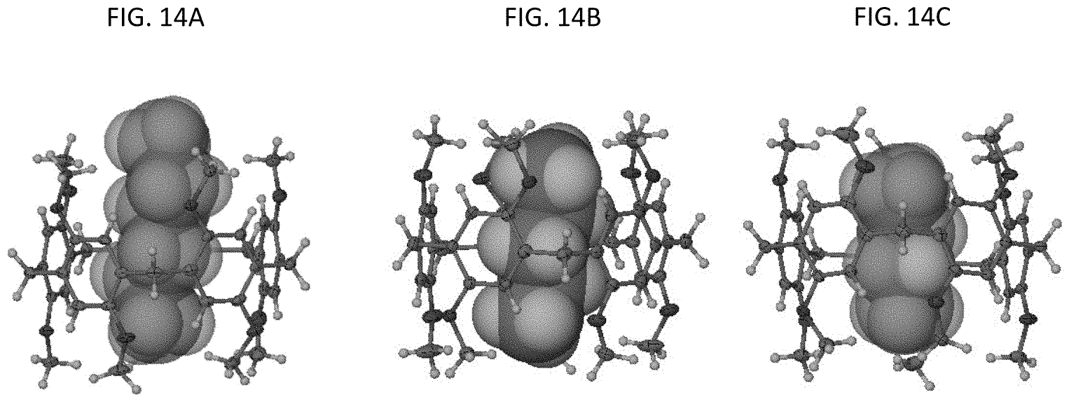

As a preliminary demonstration of the utility of guest-free DMP5 as a highly sorbent relevant to gasoline upgrading, a-DMP5 was added to each of the five hexane isomers (HEX, 2MP, 3MP, 22DMB, 23DMB) forming a series of slurries. After 10 minutes of stirring, the DMP5 solid was collected by filtration and air-dried to remove surface bound hexanes. The solid was then analyzed by PXRD to examine the solid form of DMP5. The solid DMP5 material obtained from slurries of HEX and 2MP were found to have completely converted to the β-hexane@DMP5 inclusion compounds (FIGS. 12A-12B), illustrating rapid and efficient uptake of these low-value hexane isomers, whereas the DMP5 recovered from 3MP, 22DMB and 23DMB had either converted to the guest-free α-DMP5 (3MP and 23DMB) or was a mixture of guest-free α-DMP5 and a-DMP5. In a related experiment, an equal-volume mixture of the five hexane isomers was treated with solid a-DMP5, forming a slurry. After 10 minutes of stirring the DMP5 solid was removed by filtration, dried, and the sample analyzed by GC-MS. The results, shown in FIGS. 13A-13B, show that DMP5 only absorbs/removes the low value isomers HEX and 2MP from the mixture. The data imply that DMP5 in solid form has a high affinity for the low value HEX and 2MP isomers of hexane and is entirely incapable of absorbing the high value 22DMB and 23DMB isomers. Lastly, guest-free DMP5 was evaluated for its ability to extract/absorb low value hydrocarbons directly from regular gasoline. The gasoline was treated directly with a-DMP5, forming a slurry, and, after 10 minutes of stirring, the insoluble DMP5 solid was removed by filtration and air-dried. GC-MS analysis of the solid showed the characteristic peaks for low value HEX and highly volatile PENT, illustrating the potential of guest-free DMP5 as a sorbent for the direct upgrading of refined gasoline. Along these lines, single crystals of β-HEX@DMP5, β-PENT@DMP5 and β-n-butane@DMP5 were obtained and unequivocally demonstrate the ability of DMP5 to enclathrate these normal, straight chain C₄-C₆ paraffins (FIGS. 14A-14B).

### EXPERIMENTAL

### 1.1 General Details

1,4-dimethoxybenzene was purchased from Sigma-Aldrich. Dichloromethane and methanol were purchased from BDH analytics. Acetonitrile and trifluoroacetic acid were purchased from Oakwood chemicals. CO₂ was purchased from Praxair. All deuterated solvents were purchased from Cambridge isotope laboratories.

### 1.2 Instrumental Details

¹H NMR spectra were collected on a Varian 400-MHz spectrometer at room temperature, using various delay times (5-10s) and 16-64 averaged scans. All signals were referenced to the residual solvent peak. Spectra were analyzed using MestReNova 8.1.41 software package. ¹³C NMR spectra were collected at 100 MHz on the same instrument with various delay times and averaged scans.

In-house powder X-ray diffraction (PXRD) patterns were collected in transmission mode on an APEX II DUO diffractometer with a CCD detector employing Cu—K<α> radiation (λ=1.54187 Å) generated from an IμS source. Samples analyzed by the APEX II DUO diffractometer were mounted in 0.8 mm polyimide capillaries produced by Cole-Palmer®. Synchrotron PXRD data (λ=0.412764 Å) were obtained at the Advanced Photon Source (APS) at Argonne National Laboratory. Samples were mounted in polyimide capillaries. PowDLL3, Panalytical X′Pert Highscore Plus2, Mercury, and Cambridge Structural Database (CSD) software suites were used to convert, process, analyze and compare the PXRD patterns.

Single crystal X-ray diffraction (SCXRD) data were collected on a Bruker-AXS APEX II DUO single crystal diffractometer employing Mo K<α> radiation (0.71073 Å). The crystal structures were solved by direct methods using SHELXS, and all structural refinements were conducted using SHELXL-2014-7. The program X-Seed was used as a graphical interface (GUI) for the SHELX software suite and POV-Ray, for the generation of figures.

Thermogravimetric analyses (TGA) were conducted on a TA Instruments Q5000IR TGA. 100 μL platinum pans were used for the analyses and the heating rate was maintained between 1 to 3° C./minute, as indicated. On some occasions, the TGA was attached to a Pfeiffer-Vacuum Thermostar® mass spectrometer to identify the species associated with the mass loss as analog m/z signals.

Differential scanning calorimetry (DSC) analyses were conducted in closed pans on TA Instrument Q50 DSC. The heating rates were 1-5° C./minute and the enthalpograms were integrated using TA Universal analysis 4000 V4.5A software suite. For selected samples, to allow solvent or guest molecules to escape during the heating process, pin holes were made on the lid of the DSC pans with a syringe needle before punching it on to the pan.

Gas adsorption analyses were conducted on a Quantachrome Instruments Autosorb-1 sorption analyzer for both low temperature (77 K) and room temperature collections. All samples were analyzed in a 6 mm bulb cell.

GC-MS characterization was performed on a Varian Saturn 2100T equipped with a Varian CP-8400 auto sampler using an Agilent 19091J-443, 0.25 micron, 30 m×0.35 mm column. Identification of products was carried out by comparing their retention times and electron impact (EI) mass spectra. As a general method, 1-2 μL of the analyte was added to 100 μL of 2,6-dichlorotoluene and 1 μL of the resulting solution was injected in the GC-MS using the auto sampler at 50° C. The temperature was kept constant at 50° C. for first 3 minutes and then ramped to 350° C. at a rate of 25° C./min. Filament for the mass spectra was turned off at 5 minutes mark to prevent saturation by the carrier solvent.

### 2. Synthesis

### 2. 1. Synthesis and Characterization of β-MeCN@Dimethoxypillar[5]Arene (β-Mecn@DMP5)

DMP5 was prepared as follows, similar to a previously reported method by Szumna and co-workers. In a typical synthesis, 1,4-dimethoxybenzene (11.0 g, 80.0 mmol) and paraformaldehyde (2.4 g, 80 mmol) were added to 400 ml of 1,2-dichloroethane and the mixture was degassed with nitrogen for 30 minutes, followed by the dropwise addition of trifluoroacetic acid (20 ml) under nitrogen The mixture was refluxed for 2 hours and poured into methanol to precipitate the solvated product. The crude product was dissolved in dichloromethane and an equivalent (v/v) amount of acetone was added dropwise for recrystallisation. To obtain the pure β-MeCN@DMP5 solvate, the recrystallized DMP5 product was repeatedly recrystallized from hot acetonitrile (total yield of pure β-MeCN@DMP5: 35%).

¹H NMR (400 MHz, CD₂Cl₂) δ 6.85 ppm (s, 10H, Ar—H), 3.75 ppm (s, 30H, —OCH₃), 3.72 ppm (s, 10H, —CH₂—), 1.97 ppm (s, CH₃CN).

Based on the ¹H NMR spectrum, the recrystallized solid contains at least 0.86 equivalents of MeCN per DMP5. Based on thermogravimetric analysis (TGA), the solid contains 1.05 equivalents of MeCN per DMP5. From the two techniques, the MeCN occupancy is estimated to be 0.95(1) per DMP5, referred to as β-MeCN@DMP5.

### 2. 2. Desolvation of DMP5

The desolvation of bulk β-MeCN@DMP5 was carried out by heating the material in a vacuum oven under dynamic vacuum at 235° C. for 24 hours. Depending upon the rate of cooling of the obtained melt, either crystalline (α-DMP5 or γ-DMP5) or amorphous a-DMP5 were obtained. TGA and NMR were used to confirm complete desolvation for all phases and PXRD was used to confirm the identity of the crystalline or amorphous solid forms.

¹H NMR (400 MHz, CD₂Cl₂) δ 6.84 ppm (s, 10H, Ar—H), 3.74 ppm (s, 30H, —OCH₃), 3.71 ppm (s, 10H, —CH₂—).

### 2. 3. Synthesis of Amorphous-DMP5 (a-DMP5)

Molten, desolvated DMP5 prepared in a vacuum oven was quench-cooled by rapid exposure to ambient temperature and pressure air. This procedure reproducibly led to the formation of an amorphous material, a-DMP5, with glass like appearance. The amorphous nature of the solid was confirmed by PXRD (FIG. 4C, FIG. 9A(a)). The DSC enthalpogram of a-DMP5 is shown in FIG. 4B. TGA (FIG. 4A), ¹H NMR and ¹³C NMR (FIG. 5) were used to verify the complete desolvation of the starting material.

### 2. 4. Synthesis of α-DMP5

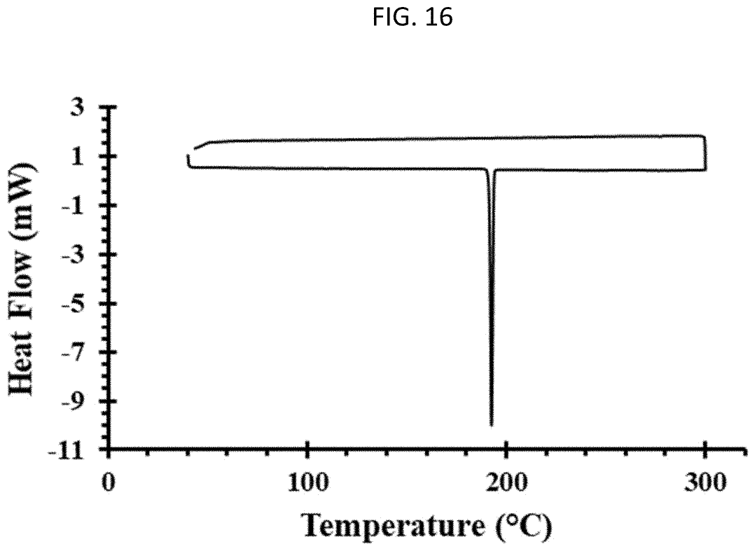

Method 1. Phase-pure α-DMP5 was obtained as a microcrystalline powder by heating β-MeCN@DMP5 at 180° C. in a glass tube, under high vacuum (facilitated with a turbo molecular pump) for 7 days. The high-temperature and high-vacuum condition resulted in partial sublimation of the DMP5 and also yielded single crystals of α-DMP5. The ¹H NMR spectrum demonstrated the absence of MeCN and that there was no detectable decomposition. Single crystal X-ray diffraction (SCXRD) (FIG. 8) and PXRD (FIG. 6) were used to identify the crystalline α-DMP5 phase. The DSC enthalpogram of α-DMP5 is shown in (FIG. 16).

Method 2. Phase-pure α-DMP5 was obtained by heating β-MeCN@DMP5 in a vacuum oven at 235° C. for 24 hours and then turning off the heating element to allow for slow (˜4 hours) cooling to room temperature under dynamic vacuum. This procedure resulted in nucleation of either α-DMP5 or γ-DMP5, seemingly at random, though α-DMP5 appeared far more often than γ-DMP5. PXRD was used to identify the crystalline form. TGA and ¹H NMR were used to verify that there was no detectable decomposition and the absence of residual solvent. The DSC of the sample obtained by this method was substantially similar to that shown in FIG. 16.

Method 3. Phase-pure α-DMP5 was obtained by heating β-MeCN@DMP5 in a vacuum oven at 190° C. for several days and then cooling the sample to room temperature under ambient pressure. PXRD was used to identify the crystalline form. TGA and ¹H NMR were used to verify that there was no detectable decomposition and the absence of residual solvent. The DSC of the sample obtained by this method was substantially similar to that shown in FIG. 16.

### 2. 5. Synthesis of γ-DMP5

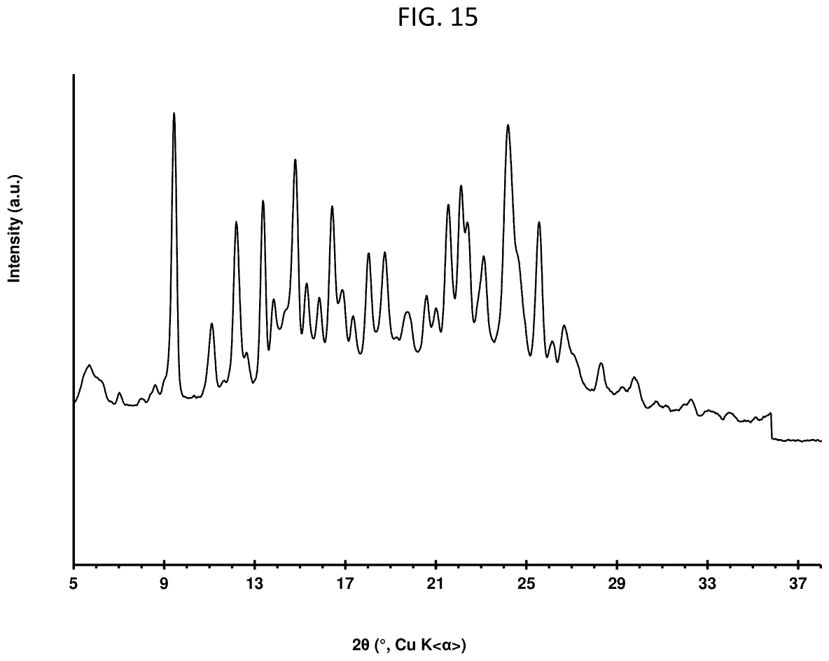

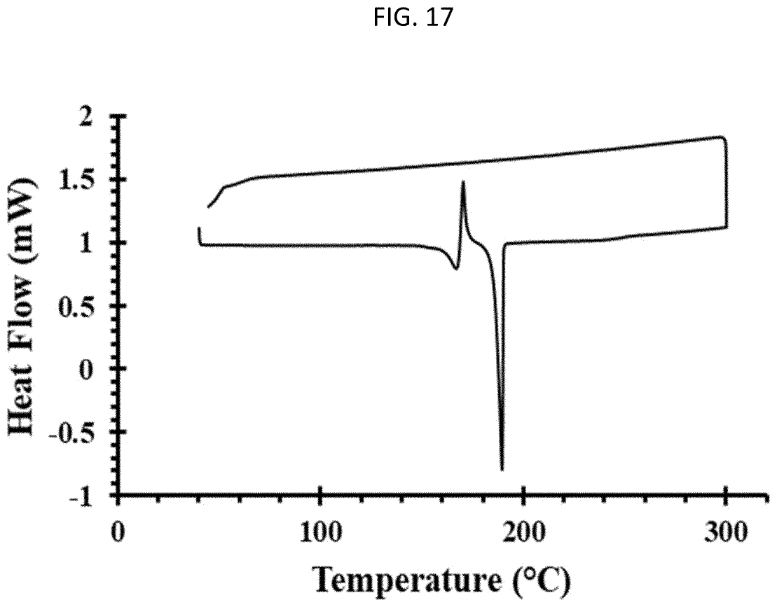

Method 1. As sample of γ-DMP5 was isolated by heating a-DMP5 to 150° C. under dynamic vacuum in a vacuum oven and then allowing the sample to cool to room temperature. PXRD was used to identify the crystalline form. TGA and ¹H NMR were used to verify that there was no detectable decomposition and the absence of residual solvent. PXRD was used to identify the crystalline form (FIG. 15). The sample was also characterized by synchrotron PXRD (FIG. 7) and the pattern was indexed to a monoclinic unit cell: a=21.62 Å, b=12.57 Å, c=15.72 Å, β=109.3°. The DSC enthalpogram is shown in FIG. 17.

Method 2. Phase-pure γ-DMP5 was obtained by heating β-MeCN@DMP5 in a vacuum oven at 235° C. for 24 hours and then turning off the heating element to allow for slow (˜4 hours) cooling to room temperature under dynamic vacuum. This procedure resulted in nucleation of either α-DMP5 or γ-DMP5, seemingly at random, though α-DMP5 appeared more often than γ-DMP5. PXRD was used to identify the crystalline form. TGA and ¹H NMR were used to verify that there was no detectable decomposition and that the sample was free of residual solvent.

Method 3. γ-DMP5 was isolated by heating a-DMP5 to 150° C. under differential scanning calorimetry (DSC) experimental conditions and cooling to room temperature. The γ-DMP5 was removed from the DSC pan. PXRD was used to identify the crystalline form.

TGA and ¹H NMR were used to verify that there was no detectable decomposition and that the sample was free of residual solvent.

### 2. 6. Synthesis of Single Crystals of β-xCO₂@DMP5 (x≤2)

A nearly saturated, warm (50° C.) solution of a-DMP5 in 2,6-dichlorotoluene was filtered and then pressurized with 34 bars of CO₂ for 7 days (while cooling to room temperature) using a custom-built, stainless steel pressure vessel. Upon release of the pressure, crystalline β-CO₂@DMP5, which rapidly begins to lose CO₂ upon expsoure to atmospheric conditions, was harvested from the solution. Repeating the experiment at different pressures revealed that the DMP5 cavities in β-xCO₂@DMP5 (x≤2) can be occupied by varying amounts of carbon dioxide (x≤2), depending upon the applied gas pressure. The crystal structure of β-CO₂@DMP5 is shown in FIG. 11A and FIG. 11B.

### 2. 7. Synthesis of Single Crystals of β-x(Xe)@DMP5 (x≤2)

A nearly saturated, warm (50° C.) solution of a-DMP5 in 2,6-dichlorotoluene was filtered and then pressurized with 20 bars of xenon for 7 days (while cooling to room temperature) using a custom-built, stainless steel pressure vessel. Upon release of the pressure, crystalline β-x(Xe)@DMP5 (x≈1.4), which loses xenon very slowly upon exposure to atmospheric conditions, was harvested from the solution. Repeating the experiments at different pressures revealed that the DMP5 cavities in β-x(Xe)@DMP5 (x≤2) can be occupied by varying amounts of xenon (x≤2), depending upon the applied xenon gas pressure. The crystal structure of β-Xeₙ@DMP5 (n≈1.4) is shown in FIG. 11C.

### 3. Special Experimental Details

### 3. 1. High Gas-Pressure Phase Conversion PXRD Experiments

A custom-built “environmental gas cell,” consisting of a glass capillary epoxied to a threaded, stainless steel valve that allows for introduction of gas pressure and subsequent sealing of the cell. Polyimide capillaries loaded with a given DMP5 form were inserted into the glass capillary of the gas cell and the cell was then loaded with the described pressure of gas. The gas cell was mounted on the APEX II Duo diffractometer using a customized goniometer designed to hold custom gas cell. The samples were kept at room temperature between successive data collections and were occasionally repressurized to ensure constant pressure in the advent of any leaks.

### 3. 2. Degassing of β-CO₂@DMP5 to Give β-xCO₂@DMP5 (x<1)

Storage of β-CO₂@DMP5 in a capped vial (non-screw) under ambient conditions allowed for slow off-gassing of β-CO₂@DMP5 to give β-xCO₂@DMP5 (x<1). To increase the rate of off-gassing, open vials of β-CO₂@DMP5 were placed in an oven at 40° C. for varying degrees of time. The CO₂ content (x) of the prepared samples of β-xCO₂@DMP5 (x<1) was determined by TGA and the and crystal form of these samples was established by approximately concurrent PXRD analysis.

### 3. 3. Selective Sorption of Paraffins/Hydrocarbons

For pure liquids: Activated a-DMP5 (70-100 mg) was added to an excess of the potential sorbate liquid hexane isomer (˜5 ml). The resulting slurries were stirred for about 10 minutes and the solid was then removed by filtration and air-dried. PXRD analysis of the solid revealed the DMP5 phase. For mixtures: Activated a-DMP5 (70-100 mg) was added to an equavolume mixture of the hexane isomers (0.4 ml each) or gasoline (˜5 ml). The resulting slurries were stirred for about 10 minutes and the solid was then removed by filtration and air-dried. For GC-MS analysis, 2-3 mg of the solid DMP5 material obtained from the slurries was dissolved in 2,6-dichlorotoulene and the solutions were analyzed for paraffin content by the GC-MS method described above.

In view of the many possible embodiments to which the principles of the disclosed invention may be applied, it should be recognized that the illustrated embodiments are only preferred examples of the invention and should not be taken as limiting the scope of the invention.

What is claimed is:

1. A composition comprising a compound of formula I: 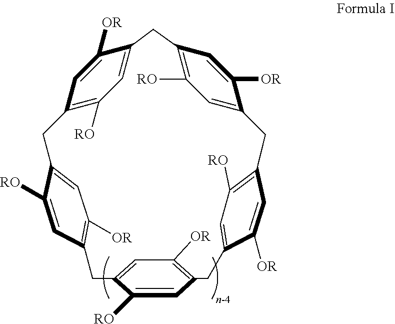
wherein n is 5, and R is methyl; and
the composition is in an essentially guest-free solid form.

2. The composition of claim 1, wherein the compound is guest-free amorphous DMP5 (a-DMP5).

3. The composition of claim 1, wherein the compound is guest-free crystalline α-DMP5.

4. The composition of claim 1, wherein the compound is guest-free crystalline γ-DMP5.

5. The composition of claim 2, wherein a room temperature PXRD pattern (Cu K<α>) of the guest-free amorphous DMP5 represents the same solid form as that represented by FIG. 4C.

6. The composition of claim 3, wherein the guest-free crystalline α-DMP5 has a unit cell of at 100(5) K as measured by X-ray diffraction of a=8.8(2) Å, b=12.8(2) Å, c=18.2(2) Å, α=96.1(9)°, β=90.9(9)°, γ=105.7(9°).

7. The composition of claim 3, wherein the guest-free crystalline α-DMP5 has a unit cell at room temperature as measured by X-ray diffraction of a=8.9(2) Å, b=12.9(2) Å, c=18.0(2) Å, α=96.6(9)°, β=90.9(9)°, γ=105.3(9)°.

8. The composition of claim 3, wherein a room temperature PXRD pattern (Cu K<α>) of the guest-free crystalline α-DMP5 represents the same solid form as that represented by FIG. 6.

9. The composition of claim 4, wherein the guest-free crystalline γ-DMP5 has a unit cell at room temperature as measured by X-ray diffraction of a=21.6(2) Å, b=12.6(2) Å, c=15.7 (2) Å, α=90.0(9)°, β=109.3(9)°, γ=90.0(9)°.

10. The composition of claim 4, wherein a PXRD pattern (Cu K<α>) of the guest-free crystalline γ-DMP5 represents the same crystal form as that represented by FIG. 15.

11. The composition of claim 4, wherein a synchrotron PXRD pattern (λ=0.412764 Å) of the guest-free crystalline γ-DMP5 represents the same crystal form as that represented by FIG. 7.

12. A composition comprising a host-guest complex, wherein the host is a compound of formula I: 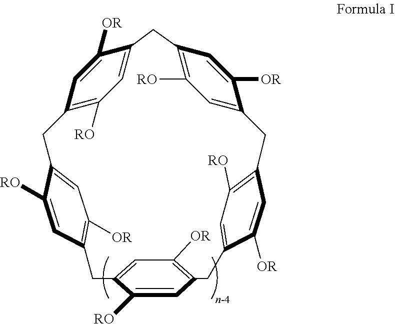
wherein n is 5, and R is methyl; and
the guest is molecules or atoms that exist as gasses at room temperature and atmospheric pressure.

13. The composition of claim 1, wherein the composition is essentially free of solvent molecules.

14. A method comprising:
exposing a sample comprising a chemical mixture to the composition of claim 1; and
selectively forming a host-guest complex between the composition of claim 1 and one or more of the components from the sample.

15. The method of claim 14, wherein the chemical mixture includes a gas component that becomes the guest in the host-guest complex and the gas is a gas molecule selected from at least one of acetylene, argon, krypton, xenon, radon, carbon dioxide, methane, ethylene, ethane, propyne, propene, propane, butanes, butenes, butadienes, fluoromethane, chloromethane, chloroethane, dimethylether, freons, gaseous fluorocarbons, methanethiol, oxygen, nitrogen, and bromomethane.

16. The method of claim 15, wherein the gas in the host-guess complex is carbon dioxide or n-butane.

17. A method comprising:
exposing a liquid petroleum mixture to the composition of claim 1; and
selectively forming a host-guest complex between the composition of claim 1 and one or more components of the liquid petroleum mixture.

18. The method of claim 17, wherein the component is n-hexane, n-pentane, n-heptane, monomethyl substituted 2-methylpentane, 3-methylpentane, or 2-methylhexane.

19. The method of claim 17, wherein the component is n-hexane, n-pentane, or 2-methylhexane.
